(19) United States
(12) Patent Application Publication
(10) Pub. No.: US 2026/0144498 A1
(43) Pub. Date: May 28, 2026

(54) Device and Method for Providing User Interface for Providing Mental Health Information

(71) Applicant: BWAVE Inc., Goyang-si Gyeonggi-do (KR)

(72) Inventor: Seung Hwan Lee, Goyang-si Gyeonggi-do (KR)

(73) Assignee: BWAVE Inc., Goyang-si Gyeonggi-do (KR)

(21) Appl. No.: 19/123,143

(22) PCT Filed: Jul. 13, 2023
(86) PCT No.: PCT/KR2023/010029

§ 371 Date: Apr. 22, 2025

(30) Foreign Application Priority Data
Oct. 24, 2022 (KR) 10-2022-0137511

(51) Int. Cl. A61B 5/00; A61B 5/024; A61B 5/384; G06T 11/10; G06T 11/26; G16H 20/70

(52) U.S. Cl. CPC A61B 5/7475; A61B 5/02405; A61B 5/384; A61B 5/4035; A61B 5/743; G06T 11/10; G06T 11/26; G16H 20/70; G06T 2200/24; G06T 2210/41

(57) ABSTRACT

The present disclosure relates to a method performed by a processor of a mental health information providing device for providing a user interface for providing mental health information, the method including obtaining brain wave data and heartbeat data of a user, calculating a brain health index representing brain activity, brain flexibility, brain intelligence, and brain balance based on the brain wave data, calculating a mental health index representing autonomic nerve activity, autonomic nerve balance, and stress based on the heartbeat data, and providing a service interface screen that graphically visualizes the calculated brain health index and mental health index.

### CROSS REFERENCE AND PRIORITY TO RELATED APPLICATIONS

This application claims the benefit of priority to: Korean Patent Application No. KR1020240057146 entitled “DEVICE AND METHOD FOR PROVIDING USER INTERFACE FOR PROVIDING MENTAL HEALTH INFORMATION,” filed on Oct. 24, 2022; and PCT Application No. PCT/KR 2023/010029 entitled “DEVICE AND METHOD FOR PROVIDING USER INTERFACE FOR PROVIDING MENTAL HEALTH INFORMATION,” filed on Jul. 13, 2023.

All the aforementioned applications are hereby incorporated by reference in their entirety.

### TECHNICAL FIELD

The present disclosure relates to a device and method for providing a user interface for mental health information providing service.

### BACKGROUND ART

As the service industry develops and the number of emotional labor workers increases, the number of people suffering from mental stress and depression is increasing. As we enter an aging society, the number of people suffering from cognitive dysfunction such as dementia is also increasing. Recently, this phenomenon has been recognized as a social problem, and various studies are being conducted to diagnose, prevent, and treat mental health to solve this problem.

For example, a technology has been disclosed that acquires frontal lobe brain wave data of the user from an electroencephalography (EEG) measuring device and measures depression severity of the user by analyzing the frontal lobe brain wave data.

However, even when mental health is diagnosed through these technologies, there is a problem that users cannot easily understand the diagnosis results that include professional terms. In addition, there is a problem that the evaluation of mental health is only valuable as an evaluation because it is not known what response to take based on the diagnosis results.

The background technology of the present disclosure has been written to facilitate understanding of the present disclosure. It should not be understood that the matters described in the background technology of the disclosure are recognized as prior art.

### DISCLOSURE

### Technical Problem

Accordingly, there is a need for a method to provide the results of mental health diagnoses, such as depression, stress-related mental health, and dementia, in a form that is easier for the general public to understand.

As a result, the inventors of the present disclosure attempted to develop a method and a device for performing the same capable of calculating result values of a plurality of indicators related to mind health and mental health using a user's biometric data and summarizing and providing readable information on mind health and mental health.

The tasks of the present disclosure are not limited to the tasks mentioned above, and other tasks not mentioned will be clearly understood by those skilled in the art from the description below.

### Technical Solution

In order to solve the above-described problem, a method performed by a processor of a mental health information providing device for providing a user interface for providing mental health information according to one embodiment of the present disclosure is provided. The method includes obtaining brain wave data and heartbeat data of a user, calculating a brain health index representing brain activity, brain flexibility, brain intelligence, and brain balance based on the brain wave data and calculating a mental health index representing autonomic nerve activity, autonomic nerve balance, and stress based on the heartbeat data, and providing a service interface screen that graphically visualizes the calculated brain health index and mental health index.

According to another aspect of the present disclosure, the service interface screen may include a region that displays an activation level of each frequency band of Delta wave, Theta wave, Low-Alpha, High-Alpha, Low-Beta, Middle-Beta, High-Beta, Gamma calculated based on the brain wave data as a graph for the brain activity.

According to still another aspect of the present disclosure, the service interface screen may include a region where the brain activation level by each frequency band is displayed in different colors in the graph for the brain activity and a normal range region is displayed on a reference axis representing the brain activation level by each frequency band.

According to still another aspect of the present disclosure, the calculating may include further includes comparing a brain activation level of each of a plurality of frequency bands with a preset reference value, and generating a brain-related diagnosis result of a user based on a comparison result, and the interface screen may include a region representing the diagnosis result in a region adjacent to the graph for the brain activity.

According to still another aspect of the present disclosure, the service interface screen may include a region that represents connection strength between the brain regions calculated based on the brain wave data as a continuous spectrum graph for the brain flexibility.

According to still another aspect of the present disclosure, the service interface screen may include a region where the connection strength is displayed at one point in the continuous spectrum corresponding to the brain flexibility, and brain network images corresponding to the reference values of the continuous spectrum are displayed.

According to still another aspect of the present disclosure, the service interface screen may include a region where a peak value of any one frequency band calculated based on the brain wave data is represented as a graph for the brain intelligence in a quadrant representing a frequency and an output value (μV²) of the frequency.

According to still another aspect of the present disclosure, the service interface screen may include a region that represents a prefrontal activation asymmetry index calculated based on the brain wave data as a semicircular scale graph for the brain balance.

According to still another aspect of the present disclosure, the service interface screen may include a region that is configured such that the prefrontal activation asymmetry index points to a point on a semicircular scale corresponding to left and right prefrontal activation levels and that behavioral characteristic information according to asymmetry on the semicircular scale is displayed at both ends.

According to still another aspect of the present disclosure, the method may further include, after the obtaining, extracting heart rate variability (HRV) data for calculating the mental health index based on the heartbeat data.

According to still another aspect of the present disclosure, the service interface screen may include a region that displays an activation level of each component of a very low frequency (VLF), a low frequency (LF), a high frequency (HF), and total power (TP) (including VLF, LF, and HF) calculated based on the heart rate variability data as a graph for the autonomic nerve activity.

According to still another aspect of the present disclosure, the service interface screen may include a region where the activation level is displayed in different colors in a graph for the autonomic nerve activity and a normal range region is displayed on a reference axis representing the activation level.

According to still another aspect of the present disclosure, the service interface screen may include a region where the diagnosis result related to the autonomic nerve activity is displayed in a region adjacent to the graph for the autonomic nerve activity.

According to still another aspect of the present disclosure, the service interface screen may include a region that displays sympathetic and parasympathetic nerve activity calculated based on the heart rate variability data as a percentage ratio in a comparison graph with respect to the autonomic nerve balance.

According to still another aspect of the present disclosure, the service interface screen may include a region where each percentage ratio in the comparison graph for the autonomic nerve balance is displayed as a bar scale, and abnormal phenomenon information according to imbalance of the sympathetic and parasympathetic nerve activity is displayed at both ends.

According to still another aspect of the present disclosure, the service interface screen may include a region that displays a stress index and stress resistance calculated based on the heart rate variability data as a continuous spectrum graph for the stress state.

According to still another aspect of the present disclosure, the service interface screen may include a region where the stress index and the stress resistance are displayed at one point in a continuous spectrum corresponding to the stress state.

According to still another aspect of the present disclosure, the method may further include, before the calculating, obtaining psychological test result data of the user, and the service interface screen may include a region that represents a numerical value corresponding to the test result data as a continuous spectrum graph for subjective psychological state.

According to still another aspect of the present disclosure, the calculating may further include generating a self-understanding value of the user based on the heart rate variability data and the psychological test result data, and the service interface screen may include a region that a continuous spectrum graph for self-understanding is placed in a region adjacent to a graph related to the mental health index, and the self-understanding value is displayed at one point in the continuous spectrum.

According to still another aspect of the present disclosure, the service interface screen may include a region where graphic objects representing diagnosis results of each of the brain health index representing the brain activity, brain flexibility, brain intelligence, and brain balance, and the mental health index representing the autonomic nerve activity, autonomic nerve balance, stress, and subjective psychological state are displayed.

In order to solve the above-described problem, a mental health information providing device according to another embodiment of the present disclosure is provided. The device includes a communication interface, a memory, and a processor operably connected to the communication interface and the memory, in which the processor is configured to obtain brain wave data and heartbeat data of a user, calculate a brain health index representing brain activity, brain flexibility, brain intelligence, and brain balance based on the brain wave data, calculate a mental health index representing autonomic nerve activity, autonomic nerve balance, and stress based on the heartbeat data, and provide a service interface screen that graphically visualizes the calculated brain health index and mental health index.

Specific details of other embodiments are included in the detailed description and drawings.

### Advantageous Effects

According to the present disclosure, it is possible to accurately calculate brain health indices such as brain activity, brain flexibility, brain intelligence, and brain balance by using brain wave data acquired from a sensor that measures brain wave signals.

In addition, according to the present disclosure, it is possible to accurately calculate mental health indices such as autonomic nerve activity, autonomic nerve balance, and stress by using heartbeat data obtained from a sensor that measures heartbeat.

In particular, the present disclosure can help with stress assessment of high-risk occupational groups, stress management of workers in the workplace, preventive medical management of mental health, and rapid evaluation (diagnosis) by medical staff by numerically expressing a person's mental and emotional state as a brain health index and a mental health index. Further, the present disclosure can be utilized as a means for preventing diseases and accidents (for example, health checkup records for insurance subscription) depending on the user's current condition.

In addition, the present disclosure can help users understand their current mental health status by simplifying and expressing the professional diagnosis region in a graph.

In addition, the present disclosure can provide users with meaningful information for real life, rather than just simple diagnosis results, by providing detailed descriptions of diagnosis contents, symptoms, treatment contents, or the like through the user interface.

The effects according to the present disclosure are not limited to those exemplified above, and more diverse effects are included in the present disclosure.

### DESCRIPTION OF DRAWINGS

FIG. 1 is a schematic diagram of a user interface providing system for providing mental health information according to one embodiment of the present disclosure.

FIG. 2 is a schematic diagram illustrating the configuration of a user device according to one embodiment of the present disclosure.

FIG. 3 is a block diagram illustrating the configuration of a mental health information providing device according to one embodiment of the present disclosure.

FIG. 4 is a schematic flowchart of a mental health information providing method according to one embodiment of the present disclosure.

FIGS. 5A, 5B, 5C, 5D, 5E, and 5F are examples of a user interface screen for providing mental health information provided through a user device according to one embodiment of the present disclosure.

### BEST MODE

The advantages and features of the present disclosure, and the methods for achieving them, will become clearer with reference to the embodiments described in detail below together with the accompanying drawings. However, the present disclosure is not limited to the embodiments disclosed below, but may be implemented in various different forms, and these embodiments are provided only to make the disclosure of the present disclosure complete and to fully inform those skilled in the art of the scope of the disclosure. In connection with the description of the drawings, similar reference numerals may be used for similar components.

In this document, the expressions “have”, “can have”, “include”, or “may include” indicate the presence of a given feature (for example, a numerical value, function, operation, or component such as a part), but do not exclude the presence of additional features.

In this document, the expressions “A or B”, “at least one of A and/or B”, or “one or more of A or/and B” can include all possible combinations of the listed items. For example, “A or B”, “at least one of A and B”, or “at least one of A or B” may all refer to (1) including at least one A, (2) including at least one B, or (3) including both at least one A and at least one B.

The expressions “first”, “second”, “first”, or “second”, or the like, used in this document can describe various components, regardless of order and/or priority, and are only used to distinguish one component from another, but do not limit the components. For example, a first user device and a second user device can represent different user devices, regardless of order or priority. For example, without departing from the scope of the rights set forth in this document, a first component can be referred to as a second component, and similarly, a second component can also be referred to as a first component.

When it is stated that a component (for example, a first component) is “(operatively or communicatively) coupled with/to” or “connected to” another component (for example, a second component), it should be understood that the component can be directly coupled to the other component, or can be connected via another component (for example, a third component). Conversely, when it is stated that a component (for example, a first component) is “directly coupled with” or “directly connected to” another component (for example, a second component), it should be understood that no other component (for example, a third component) exists between the component and the other component.

The expression “configured (or set) to” as used herein may be used interchangeably with, for example, “suitable for”, “having the capacity to”, “designed to”, “adapted to”, “made to”, or “capable of”. Moreover, the term “configured (set) to” does not necessarily mean something is “specifically designed to” in terms of hardware. Instead, in some contexts, the expression “a device configured to” can mean that the device is “capable of” doing something together with other devices or components. For example, the phrase “a processor configured (or set) to perform A, B, and C” can mean a dedicated processor (for example, an embedded processor) to perform those operations, or a generic-purpose processor (for example, a CPU or an application processor) that can perform those operations by executing one or more software programs stored in a memory device.

The terms used in the present document are only used to describe specific embodiments and may not be intended to limit the scope of other embodiments. The singular expression may include the plural expression unless the context clearly indicates otherwise. The terms used herein, including technical or scientific terms, may have the same meaning as commonly understood by a person of ordinary skill in the art described in this document. Among the terms used in this document, terms defined in general dictionaries may be interpreted as having the same or similar meaning in the context of the related technology, and shall not be interpreted in an ideal or excessively formal meaning unless explicitly defined in this document. In some cases, even if a term is defined in this document, it cannot be interpreted to exclude the embodiments of this document.

The individual features of the various embodiments of the present disclosure may be partially or wholly combined or combined with each other, and as can be fully understood by those skilled in the art, various technical connections and operations are possible, and each embodiment may be implemented independently of each other or may be implemented together in a related relationship.

For clarity in the interpretation of this specification, the terms used in this specification are defined below.

As used herein, the term “brain wave data” may refer to EEG (electroencephalogram) signal values recorded by a sensor that detects brain waves. More specifically, the brain wave data may be a brain wave signal of a positive potential response that appears after a stimulus of a certain intensity.

Meanwhile, since the brain wave data may be a signal or a signal value obtained from a sensor, the brain wave data may be interpreted in the same meaning as sensor/sensing data.

According to a feature of the present disclosure, the brain wave data may include brain wave data measured from at least one electrode of Fp1, Fp2, F3, Fz, F4, F8, T7, C3, C4, Cz, T8, P7, P3, Pz, P4, P8, 01, 02, FCz, TP9, TP10, Oz, A Fz, F7, Fpz, AF7, AF3, AF4, AF8, F9, F5, F1, F2, F6, F10, FT9, FT7, FC5, FC3, FC1, FC2, FC4, FC6, FT8, FT10, C5, C1, C2, C6, TP7, CP5, CP3, CP1, CPz, CP2, CP4, CP6, TP8, P9, P5, P1, P2, P6, P10, PO9, PO7, PO3, POz, PO4, PO8, PO10, O9, Iz, O10, F11, F12, FT11, FT12, TP11, TP12, PO11, PO12, P11, P12, I11, I12 and IIz.

According to a feature of the present disclosure, the brain wave data may be brain wave data obtained in a resting state in which no stimulation is applied to an object, but is not limited thereto.

As used herein, the term “heartbeat data” may refer to electrocardiogram (ECG), blood pressure photoplethysmography (PPG) signal values recorded by a sensor that detects a heartbeat. More specifically, the heartbeat data may be data representing changes in, or variability in the time interval between heartbeats (between peaks).

Hereinafter, the present disclosure will be described in detail by explaining preferred embodiments of the present disclosure with reference to the attached drawings.

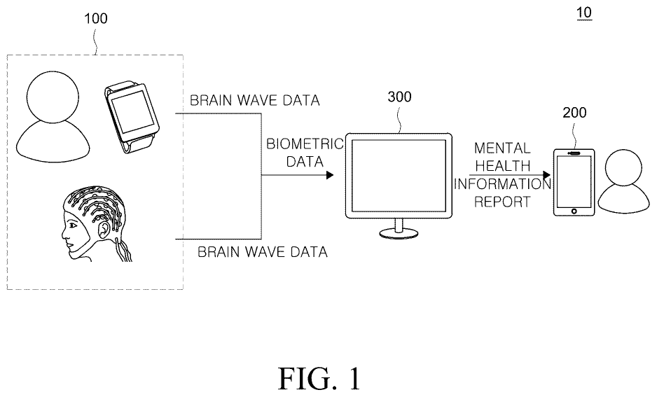

FIG. 1 is a schematic diagram of a user interface providing system for providing mental health information according to one embodiment of the present disclosure.

Referring to FIG. 1, a user interface providing system 10 (hereinafter referred to as “mental health information system 10”) for providing mental health information according to one embodiment of the present disclosure may include a sensing device 100 for measuring a user's biometric data, a user device 200 carried by the user, and a user interface providing device (hereinafter referred to as “mental health information providing device 300”) for providing the user's brain and mental health information in the form of a user interface.

The sensing device 100 is a device capable of acquiring the user's biometric data, and may include various devices capable of measuring the user's heartbeat depending on the type of biometric data. For example, the sensing device 100 may be a wearable device worn on the user's wrist, and may acquire the user's electrocardiogram data through the wearable device. For another example, the sensing device 100 may correspond to the user device 200, and may acquire the user's pulse data (photoplethysmography) based on the change in blood flow of the fingertips through the user device 200. For another example, the sensing device 100 may be a patch-type device that adheres closely to the user's scalp, and may acquire the user's brain wave data through this.

In one embodiment of the present disclosure, biometric data related to the user's mental health may include data capable of extracting temporal variations of heartbeats rather than changes in heartbeat, that is, all types of biometric data capable of evaluating the balance between the sympathetic and parasympathetic nerves of the autonomic nervous system and the activity level of each.

The user device 200 is a device possessed by the user that can check mental health information, and may include a smartphone, a tablet PC, a PC, a laptop, or the like. Specifically, the user device 200 may transmit biometric data (brain wave data and heartbeat data) measured by the sensing device 100 to the mental health information providing device 300, and may receive and output a user interface including a brain health index and a mental health index visualized as a graph from the mental health information providing device 300.

In various embodiments, the user device 200 may output a psychological test interface for measuring the user's psychological state, such as depression or anxiety. For example, the user device 200 may receive a questionnaire for the user's psychological test from the mental health information providing device 300 and obtain the user's response thereto.

The mental health information providing device 300 may include a general-purpose computer, a laptop, a data server, or the like that performs various operations to calculate the brain health index and mental health index of the user based on the biometric data (brain wave data and heartbeat data) measured by a sensing device 100 and provide the calculated indices to the user in the form of a mental health information report. Specifically, the mental health information providing device 300 may refine the brain wave data and heartbeat data into data forms for calculating the brain health index and the mental health index. For example, the mental health information providing device 300 may filter the brain wave data to extract brain activity data for a specific frequency, and as another example, the mental health information providing device 300 may extract heart rate variability (HRV) data from the heartbeat data.

The mental health information providing device 300 may provide the user interface that outputs information related to the brain health index and the mental health index. For example, the mental health information providing device 300 may provide a mobile or web application/program that can be installed or executed by the user device 200, and may visually provide the user's mental health information through the mobile or web application/program.

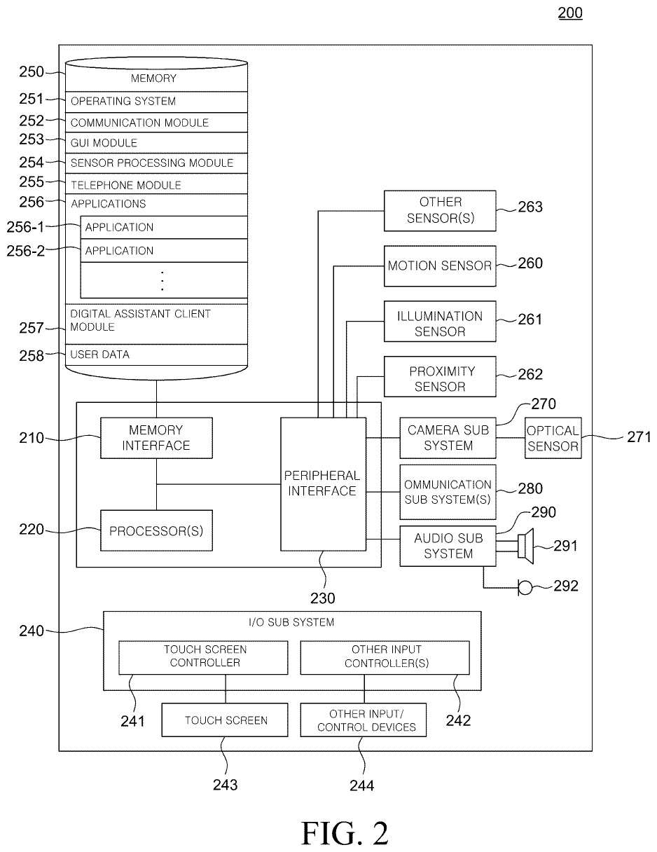

So far, the mental health information system 1000 according to one embodiment of the present disclosure has been described, and below, the user device 200 that outputs a user interface screen for the mental health information will be described with reference to FIG. 2.

FIG. 2 is a schematic diagram illustrating the configuration of the user device according to one embodiment of the present disclosure.

Referring to FIG. 2, the user device 200 may include a memory interface 210, one or more processors 220, and a peripheral interface 230. Various components within the user device 200 may be connected by one or more communication buses or signal lines.

The memory interface 210 is connected to the memory 250 and may transmit various data to the processor 220. Here, the memory 250 can include at least one type of storage medium among a flash memory type, a hard disk type, a multimedia card micro type, a card type memory (for example, an SD or XD memory, or the like), a RAM, an SRAM, a ROM, an EEPROM, a PROM, a network storage, a cloud, and a blockchain database.

In various embodiments, the memory 250 may store the user's biometric data (brain wave data and heartbeat data) and the user's psychological test result data. In addition, the memory 250 may store each configuration of a user interface for outputting the user's brain health index and mental health index calculated based on the biometric data and psychological test result data.

In various embodiments, the memory 250 may store at least one of an operating system 251, a communication module 252, a graphical user interface module GUI 253, a sensor processing module 254, a telephone module 255, and an application module 256. Specifically, the operating system 251 may include instructions for processing basic system services and instructions for performing hardware operations. The communication module 252 may communicate with at least one of other ore or more devices, computers, and servers. The graphical user interface module GUI 253 may process a graphical user interface. The sensor processing module 254 may process sensor-related functions (for example, processing voice input received via one or more microphones 292). The telephone module 255 may process telephone-related functions. The application module 256 may perform various functions of a user application, such as electronic messaging, web browsing, media processing, navigation, imaging, and other processing functions. In addition, the user device 100 may store one or more software applications 256-1 and 256-2 (for example, an application for providing mental health information) associated with one type of service in the memory 250.

In various embodiments, the memory 250 may store a digital assistant client module 257 (hereinafter, DA client module), and thereby store instructions for performing client-side functions of the digital assistant and various user data 258 (for example, user-customized vocabulary data, preference data, other data such as the user's electronic address book, or the like).

Meanwhile, the DA client module 257 may obtain the user's voice input, text input, touch input, and/or gesture input through various user interfaces (for example, I/O subsystem 240) provided in the user device 200.

In addition, the DA client module 257 may output data in audiovisual and tactile forms. For example, the DA client module 257 may output data consisting of a combination of at least two or more of voice, sound, notification, text message, menu, graphic, video, animation, and vibration. In addition, the DA client module 257 may communicate with a digital assistant server (not illustrated) using a communication subsystem 280.

In various embodiments, the DA client module 257 may collect additional information about the surroundings of the user device 200 from various sensors, subsystems, and peripheral devices to construct a context associated with the user input. For example, the DA client module 257 may provide context information along with the user input to a digital assistant server to infer the user's intent. Here, the context information that may accompany the user input may include sensor information, such as lighting, ambient noise, ambient temperature, images of the surroundings, videos, or the like. As another example, the context information may include a physical state (for example, device orientation, device position, device temperature, power level, speed, acceleration, motion patterns, cellular signal strength, or the like) of the user device 200. As yet another example, the context information may include information (for example, processes running on the user device 200, installed programs, past and present network activity, background services, error logs, resource usage, or the like) related to software state of the user device 200.

In various embodiments, the memory 250 may include additional or deleted instructions, and further, the user device 200 may include additional configurations other than those illustrated in FIG. 2, or may exclude some configurations.

The processor 220 may control the overall operation of the user device 200 and execute various commands to implement the user interface that provides the mental health information by running an application or program stored in the memory 250.

The processor 220 may correspond to a computational device such as a central processing unit (CPU) or an application processor (AP). In addition, the processor 220 may be implemented in the form of an integrated chip IC such as a System on Chip (SoC) in which various computational devices that perform machine learning, such as a neural processing unit (NPU), are integrated.

In various embodiments, the processor 220 may obtain the user's biometric data (brain wave data and heartbeat data), output the user interface screen graphically visualizing the mental health index and brain health index calculated based on the biometric data, and provide various contents (detailed description of diagnosis, symptoms, treatment contents) related to the mental health index and brain health index according to the user's interaction.

The peripheral interface 230 may be connected to various sensors, subsystems, and peripheral devices to provide data so that the user device 200 can perform various functions. Here, it can be understood that the user device 200 performs a certain function as being performed by the processor 220.

The peripheral interface 230 may receive data from a motion sensor 260, a light sensor (illumination sensor) 261, and a proximity sensor 262, through which the user device 200 may perform orientation, light, and proximity detection functions, or the like. For another example, the peripheral interface 230 may receive data from other sensors 263 (positioning system-GPS receiver, temperature sensor, biometric sensor), through which the user device 200 may perform functions related to the other sensors 263.

In various embodiments, the user device 200 may include a camera subsystem 270 connected to the peripheral interface 230 and an optical sensor 271 connected thereto, which enables the user device 200 to perform various photographing functions, such as taking pictures and recording video clips.

In various embodiments, the user device 200 may include a communication subsystem 280 coupled with a peripheral interface 230. The communication subsystem 280 may include one or more wired/wireless networks and may include various communication ports, radio frequency transceivers, and optical transceivers.

In various embodiments, the user device 200 includes an audio subsystem 290 coupled to the peripheral interface 230, the audio subsystem 290 including one or more speakers 291 and one or more microphones 292, such that the user device 200 may perform voice-activated functions, such as voice recognition, voice replication, digital recording, and telephony.

In various embodiments, the user device 200 may include an I/O subsystem 240 coupled with the peripheral interface 230. For example, the I/O subsystem 240 may control a touch screen 243 included in the user device 200 via a touch screen controller 241. For example, the touch screen controller 241 may detect a user's contact and movement or cessation of contact and movement using any one of a plurality of touch sensing technologies, such as capacitive, resistive, infrared, surface acoustic wave technology, proximity sensor array, or the like. In another example, the I/O subsystem 240 may control other input/control devices 244 included in the user device 200 via other input controller(s) 242. As an example, the other input controller(s) 242 may control one or more buttons, rocker switches, thumb-wheels, infrared ports, USB ports, and pointer devices such as a stylus.

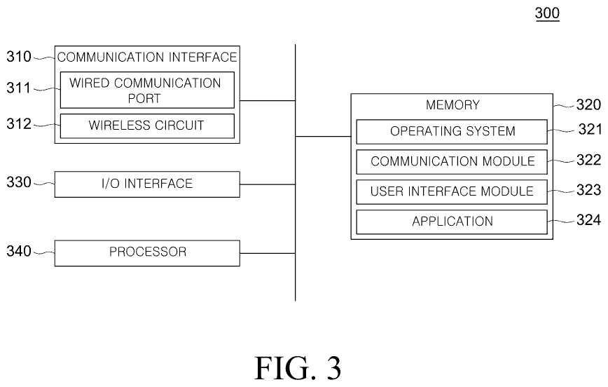

So far, the user device 200 according to one embodiment of the present disclosure has been described, and below, the mental health information providing device 300 that provides a service of visually refining the mental health information will be described with reference to FIGS. 3 to 5F.

FIG. 3 is a block diagram illustrating the configuration of the mental health information providing device according to one embodiment of the present disclosure.

Referring to FIG. 3, the mental health information providing device 300 may include a communication interface 310, a memory 320, an I/O interface 330, and a processor 340, and each component may communicate with each other through one or more communication buses or signal lines.

The communication interface 310 may be connected to the sensing device 100 and the user device 200 via a wired/wireless communication network to exchange data. For example, the communication interface 310 may receive biometric data about a specific user from the sensing device 100 and the user device 200. As another example, the communication interface 310 may transmit a user interface that graphically visualizes mental health information to the user device 200.

Meanwhile, the communication interface 310 that enables transmission and reception of such data includes a wired communication port 311 and a wireless circuit 312, in which the wired communication port 311 may include one or more wired interfaces, for example, Ethernet, a universal serial bus USB, FireWire, or the like. In addition, the wireless circuit 312 may transmit and receive data with an external device via an RF signal or an optical signal. In addition, the wireless communication may use at least one of a plurality of communication standards, protocols, and technologies, for example, GSM, EDGE, CDMA, TDMA, Bluetooth, Wi-Fi, VoIP, and Wi-MAX, or any other suitable communication protocol.

The memory 320 may store various data used in the mental health information providing device 300. For example, the memory 320 may store user-specific biometric data (brain wave data and heartbeat data), brain health index and mental health index distribution results of users, or the like.

In various embodiments, the memory 320 may include a volatile or nonvolatile storage medium capable of storing various data, commands, and information. For example, the memory 320 may include at least one type of storage medium among a flash memory type, a hard disk type, a multimedia card micro type, a card type memory (for example, an SD or XD memory, or the like), a RAM, an SRAM, a ROM, an EEPROM, a PROM, a network storage, a cloud, and a blockchain database.

In various embodiments, the memory 320 may store configurations of at least one of an operating system 321, a communication module 322, a user interface module 323, and one or more applications 324.

The operating system 321 (for example, embedded operating systems such as LINUX, UNIX, MAC OS, WINDOWS, VxWorks, or the like) may include various software components and drivers to control and manage general system operations (for example, memory management, storage device control, power management, or the like) and may support communication between various hardware, firmware, and software components.

The communication module 323 may support communication with other devices through the communication interface 310. The communication module 220 may include various software components for processing data received by the wired communication port 311 or wireless circuit 312 of the communication interface 310.

The user interface module 323 may receive a user's request or input from a keyboard, touch screen, mouse, microphone, or the like through an I/O interface 330 and provide the user interface on the display.

The application 324 may include a program or module configured to be executed by one or more processors 340. Here, the application for providing the mental health information may be implemented on a server farm.

The I/O interface 330 may connect at least one of input/output devices (not illustrated) of the mental health information providing device 300, such as a display, a keyboard, a touch screen, and a microphone, to the user interface module 323. The I/O interface 330 may receive user input (for example, voice input, keyboard input, touch input, or the like) together with the user interface module 323 and process the command according to the received input.

The processor 340 is connected to the communication interface 310, the memory 320, and the I/O interface 330 to control the overall operation of the mental health information providing device 300, and may perform various commands for extracting standard plane information from an ultrasound image through an application or program stored in the memory 320.

The processor 340 may correspond to a computational device such as a central processing unit (CPU) or an application processor (AP). In addition, the processor 340 may be implemented in the form of an integrated chip (IC) such as a system on chip (SOC) in which various computational devices are integrated. Alternatively, the processor 340 may include a module for calculating an artificial neural network model such as a neural processing unit (NPU).

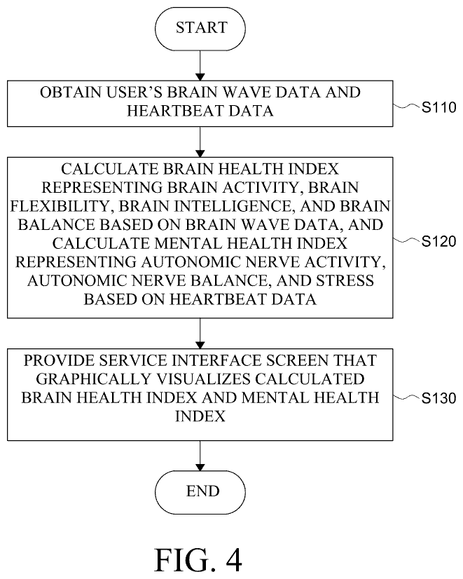

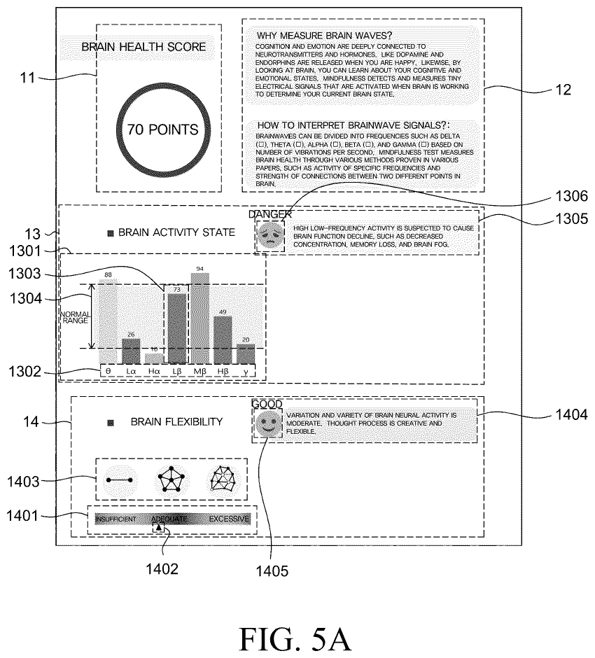

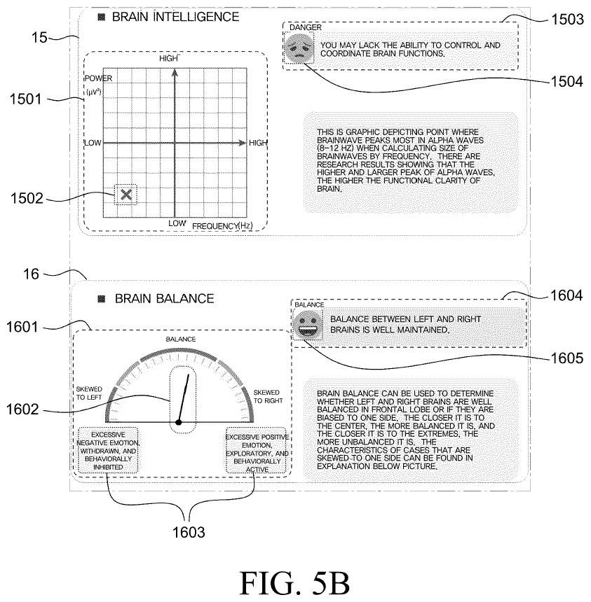

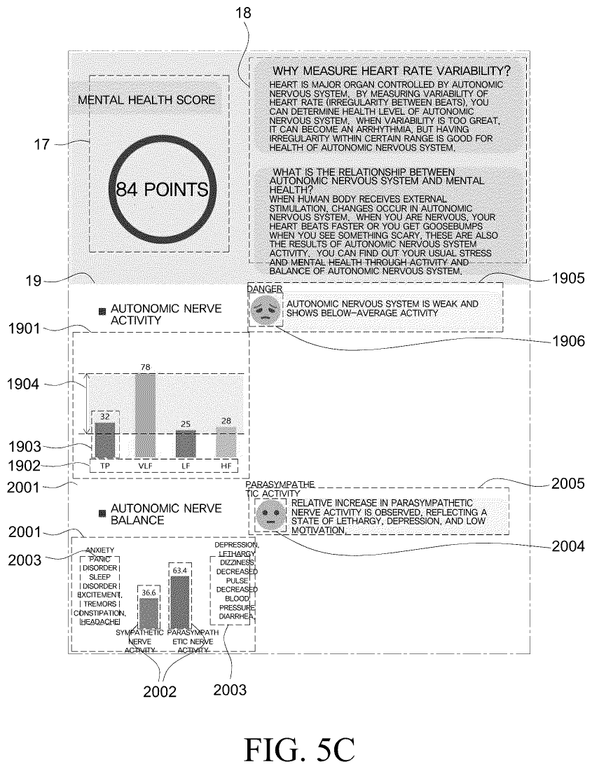

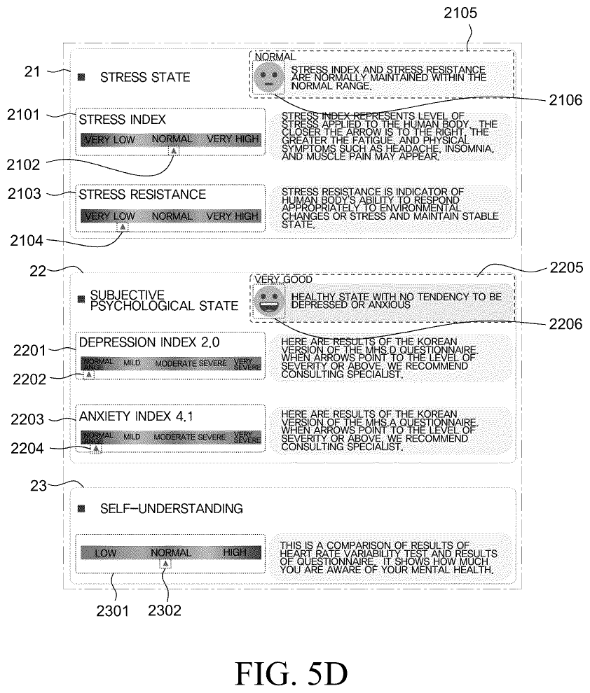

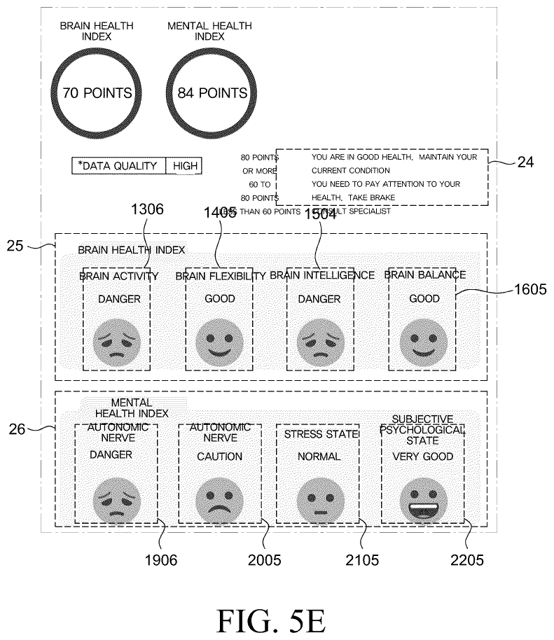

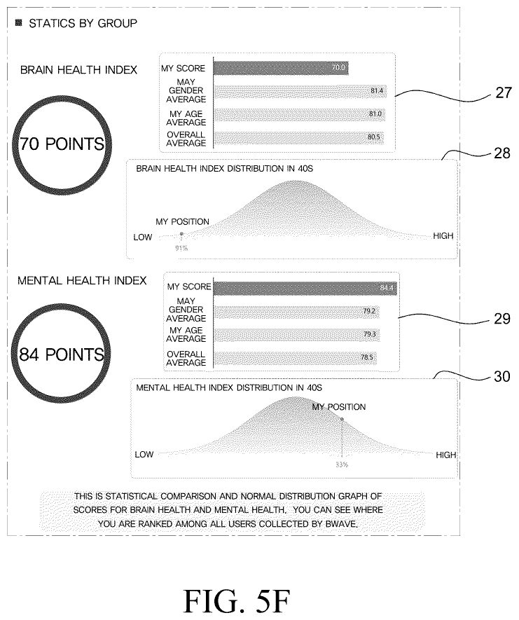

Hereinafter, with reference to FIGS. 4 to 5F, a method for providing the user interface for providing the mental health information by the processor 340 of the mental health information providing device 300 will be described.

FIG. 4 is a schematic flowchart of a method for providing the mental health information according to one embodiment of the present disclosure.

Referring to FIG. 4, the processor 340 may obtain the user's brain wave data and heartbeat data (S110). Specifically, the processor 340 may obtain the user's heartbeat data from the user device 200. The processor 340 may obtain the user's brain wave data from the sensing device 100.

In various embodiments, the processor 340 may further obtain the user's psychological test result data from the user device 200. Specifically, the processor 340 may provide the user device 200 with a psychological test interface including questions for the user's psychological test, thereby obtaining data for determining the user's psychological state, such as depression or anxiety.

In various embodiments, the processor 340 may extract brain activity data of the user for any one region based on the user's brain wave data. For example, the processor 340 may obtain brain activity data divided into a specific frequency region, for example, a delta wave of 1 to 4 Hz (a theta wave of δ4 to 8 Hz (a low-alpha wave (Lα) of θ8 to 10 Hz), a high-alpha wave (Hα) of 10 to 12 Hz, a low-beta wave (Lβ) of 12 to 15 Hz, a middle-beta wave (Mβ) of 15 to 20 Hz, a high-beta wave (Hβ) of 20 to 30 Hz, and a gamma wave (γ) of 30 to 50 Hz), as data for calculating the brain health index.

In various embodiments, the processor 340 may extract heart rate variability (HRV) data based on the user's heartbeat data. More specifically, since the heart rate variability is an indicator that may predict cardiovascular function in response to stress or anxiety, the processor 340 may extract values corresponding to components of a very low frequency (VLF) of 0 to 0.04 Hz, a low frequency (LF) of 0.04 to 0.15 Hz, and a high frequency (HF) of 0.15 to 0.4 Hz, and TP (total power; sum of HF, LF, VLF, or the like) from the heartbeat data. Here, the HF component is an indicator of parasympathetic nervous system activity and the activity level thereof changes depending on breathing, the LF component is an indicator of sympathetic nervous system activity and represents cognitive load for information processing, and the VLF component is related to body temperature regulation.

After Step S110, the processor 340 may calculate the brain health index representing the brain activity, brain flexibility, brain intelligence, and brain balance based on the brain wave data, and may calculate the mental health index representing the autonomic nerve activity, autonomic nerve balance, and stress based on the heartbeat data (S120). More specifically, the processor 340 may calculate the activation level for each frequency band from the brain activity data extracted from the brain wave data. The processor 340 may calculate the activation level for each frequency band from the heart rate variability data extracted from heartbeat data. In addition, the processor 340 may calculate the strength between a plurality of nodes constituting a brain network based on the brain activity data for each of the plurality of regions. The processor 340 may calculate normalized LF, normalized HF, LF/HF ratio, or the like based on the heart rate variability data.

After Step S120, the processor 340 may provide a service interface screen that graphically visualizes the calculated brain health index and mental health index (S130). That is, the processor 340 may visually represent the brain activity, brain flexibility, brain intelligence, brain balance, autonomic nerve activity, autonomic nerve balance, and stress as illustrated in FIGS. 5A to 5F.

FIGS. 5A, 5B, 5C, 5D, 5E, and 5F are examples of the user interface screen for providing the mental health information through the user device according to one embodiment of the present disclosure.

Referring to FIG. 5A, the service interface screen visualizing the brain health index may include a graphic object 11 representing the average score values of the brain activity, brain flexibility, brain intelligence, and brain balance, respectively, and an explanation region 12 explaining the basis of brain wave measurement and the method of calculating each score.

The service interface screen visualizing the brain health index may include the region 13 that illustrates the result of calculating the user's brain activity. Specifically, the service interface screen may include a region that represents an activation level 1303 for each frequency band 1302 of delta wave, theta wave, low-alpha, high-alpha, low-beta, middle-beta, high-beta, and gamma calculated based on the brain wave data (brain activity data) as a graph 1301 for brain activity. In the graph 1301 for brain activity, the activation level 1303 for each frequency band 1302 is displayed in different colors to improve visibility, and the meaning of each indicator may be displayed in one region. In addition, a normal range region 1304 is displayed on the reference axis representing the brain activation level in the graph 1301 for brain activity, so that the user can recognize that the activation level 1303 for each frequency band 1302 is not relative.

In various embodiments, the processor 340 may compare the brain activation level of each of the plurality of frequency bands with the reference value preset for each frequency band, and generate the diagnosis result related to the user's brain activity according to the comparison result. Accordingly, the service interface screen visualizing the brain health index may include a region 1305 representing the diagnosis result in a region adjacent to the graph 1301 for the brain activity. In particular, the processor 340 may determine which of the five classification items (danger/caution/normal/good/very good) the brain activity falls into based on the comparison result, and include a graphic object 1306 representing the diagnosis result on the service interface screen.

In addition, the service interface screen visualizing the brain health index may include a region 14 representing the user's brain flexibility calculation result. Specifically, the service interface screen may include a region representing the connection strength between brain regions calculated based on the brain activity data as a continuous spectrum graph 1401 for the brain flexibility. The continuous spectrum for the brain flexibility may be displayed in different colors according to the degree of flexibility (insufficient˜adequate˜excessive). The user's brain flexibility calculation result (connection strength) 1402 may be displayed at one point. In addition, a brain network image 1403 corresponding to reference values (insufficient/adequate/excessive) in the continuous spectrum for the brain flexibility may be displayed.

In various embodiments, the processor 340 may generate a brain flexibility-related diagnosis result based on the calculated result value for the brain flexibility. Accordingly, the service interface screen visualizing the brain health index may include a region 1404 representing the diagnosis result in a region adjacent to the continuous spectrum graph 1401 for the brain flexibility. In particular, the processor 340 may determine which of the five classification items (danger/caution/normal/good/very good) the calculated result value for brain flexibility belongs to, and may include a graphic object 1405 representing the diagnosis result in the service interface screen.

Referring to FIG. 5B, a service interface screen visualizing the brain health index may include a region 15 illustrating a result of calculating a user's brain intelligence. Specifically, the service interface screen may include a region where a peak value 1502 for any one frequency band calculated based on the brain wave data (brain activity data) in a quadrant illustrating a frequency (Hz) and an output value (μV²) of the frequency is represented as a graph 1501 for the brain intelligence. In particular, the processor 340 may calculate a peak value in an alpha wave (α) of 8 to 12 Hz, display the peak value on the service interface screen, and generate a diagnosis result based on the value. Accordingly, the service interface screen visualizing the brain health index may include a region 1503 representing the diagnosis result in the region adjacent to the graph 1501 for the brain intelligence. In particular, the processor 340 may determine which of the five classification items (danger/caution/normal/good/very good) the calculated result value for brain intelligence falls into, and may include a graphic object 1504 representing the diagnosis result on the service interface screen.

The service interface screen visualizing the brain health index may include a region 16 that represents the user's brain balance calculation result. Specifically, the service interface screen may include a region that represents the prefrontal activation asymmetry index 1602 calculated based on the brain wave data as a semicircular scale graph 1601 for the brain balance. The semicircular scales for the brain balance correspond to the degrees of left and right prefrontal activation, respectively, and these may be displayed in different colors according to the degrees of asymmetry. The user's prefrontal activation asymmetry index 1602 may point to a point of the semicircular scale graph 1601, and the user's behavioral characteristic information 1603 according to the asymmetry may be displayed at both ends. For example, when the prefrontal lobe is skewed to the left, the user may be represented with the characteristics of “excessive negative emotion, withdrawn, and behaviorally inhibited”, and when the prefrontal lobe is skewed to the right, the user may be represented with the characteristics of “excessive positive emotion, exploratory, and behaviorally active”.

In various embodiments, the processor 340 may generate the diagnosis result based on the brain balance calculation result. Accordingly, the service interface screen visualizing a brain health index may include a region 1604 representing a diagnosis result in a region adjacent to a semicircular scale graph 1601 for the brain balance. In particular, the processor 340 may determine which of the five classification items (danger/caution/normal/good/very good) the calculation result for the brain balance falls into, and may include a graphic object 1605 representing the diagnosis result in the service interface screen.

Referring to FIG. 5C, the service interface screen visualizing the mental health index may include a graphic object 17 representing the average score values of autonomic nerve activity, autonomic nerve balance, and stress, and an explanation region 18 explaining the basis for measuring heartbeat (heart rate variability) and the method for calculating each score.

The service interface screen visualizing the mental health index may include a region 19 that illustrates the result of calculating the user's autonomic nerve activity. Specifically, the service interface screen may include a region that represents the activation level 1903 of each component of a very low frequency (VLF), a low frequency (LF), a high frequency (HF), and a total power (TF) (including VLF, LF, and HF) calculated based on the heart rate variability data as a graph 1901 for autonomic nerve activity. In the graph 1901 for the autonomic nerve activity, the activation level 1903 of each component 1902 may be displayed in different colors to improve visibility, and the meaning of each indicator may be displayed in one region. In addition, a normal range region 1904 may be displayed on a reference axis representing the activation level of each component in the graph 1901 for the autonomic nerve activity, to make the user aware that the activation level 1903 of each frequency component 1902 is not relative.

In various embodiments, the processor 340 may generate a diagnosis result related to autonomic nerve activation based on the activation level for each frequency component. Accordingly, the service interface screen visualizing the mental health index may include the region 1905 representing the diagnosis result in the region adjacent to the graph 1901 for the autonomic nerve activity. In particular, the processor 340 may determine which of the five classification items (danger/caution/normal/good/very good) the autonomic nerve activity falls into, and include a graphic object 1906 representing the diagnosis result in the service interface screen.

In addition, the service interface screen visualizing the mental health index may include the region 20 that illustrates the result of calculating the user's autonomic nerve balance. Specifically, the service interface screen may include a region that illustrates the sympathetic and parasympathetic nerve activity calculated based on the heart rate variability data as a percentage ratio 2002 in a comparison graph 2001 for the autonomic nerve balance. In the comparison graph 2001 for the autonomic nerve balance, each percentage ratio 2002 is displayed as a bar scale, and abnormal phenomenon information 2003 according to the imbalance of the sympathetic and parasympathetic nerve activity may be displayed at both ends. For example, when the sympathetic nerve activity is biased, the user may experience characteristics such as “anxiety, panic disorder, sleep disorder, excitement, tremors, constipation, and headache”, and when the parasympathetic nerve activity is biased, the user may experience characteristics such as “depression, lethargy, dizziness, decreased pulse, decreased blood pressure, diarrhea, and edema”.

In various embodiments, the processor 340 may generate the diagnosis result related to the autonomic nerve balance based on the calculation results of the sympathetic and parasympathetic nerve activities. Accordingly, the service interface screen visualizing the mental health index may include the comparison graph 2001 for the autonomic nerve balance and a region 2005 representing the diagnosis result in an adjacent region. In particular, the processor 340 may determine which of the five classification items (danger/caution/normal/good/very good) the calculation ratio of the sympathetic and parasympathetic nerve activities belongs to, and may include a graphic object 2004 representing the diagnosis result in the service interface screen.

Referring to FIG. 5D, the service interface screen visualizing the mental health index may include a region 21 that represents a result of calculating a user's stress state. Specifically, the service interface screen may include a region that represents the stress index and the stress resistance calculated based on the heart rate variability data, respectively, as continuous spectrum graphs 2101 and 2103 for the stress state. The continuous spectrum for the stress index and the stress resistance may be displayed in different colors according to the level (very low˜normal˜very high), and the user's stress index 2102 and stress resistance 2104 may be displayed at one point.

In various embodiments, the processor 340 may generate the diagnosis result related to the stress state based on the calculation results for the stress index and the stress resistance. Accordingly, the service interface screen visualizing the mental health index may include a region 2105 representing the diagnosis result in the region adjacent to the continuous spectrum graphs 2101 and 2103 for the stress state. In particular, the processor 340 may determine which of the five classification items (danger/caution/normal/good/very good) the calculation result for the stress state belongs to, and may include a graphic object 2106 representing the diagnosis result in the service interface screen.

In addition, the service interface screen visualizing the mental health index may include regions 22 and 23 representing the user's subjective psychological state results. Specifically, the service interface screen may include a region representing the depression index and the anxiety index, which are calculated in response to the user's psychological test result data, as continuous spectrum graphs 2201 and 2203 for the subjective psychological state, respectively. The continuous spectrum for the depression index and the anxiety index may be displayed in different colors according to the degree (normal range˜mild˜moderate˜severe˜very severe), and the user's depression index 2202 and anxiety index 2204 may be displayed at one point.

In various embodiments, the processor 340 may generate a diagnosis result related to a subjective psychological state based on the calculation results for the depression index and the anxiety index. Accordingly, the service interface screen visualizing the mental health index may include a region 2205 representing the diagnosis result in the region adjacent to continuous spectrum graphs 2201 and 2203 for the subjective psychological state. In particular, the processor 340 may determine which of the five classification items (danger/caution/normal/good/very good) the calculation result for the subjective psychological state belongs to, and may include a graphic object 2206 representing the diagnosis result in the service interface screen.

In various embodiments, the processor 340 may generate a user's self-understanding value based on the heart rate variability data and the psychological test result data. Accordingly, the service interface screen visualizing the mental health index may include a continuous spectrum graph 2301 for self-understanding divided into levels (low˜normal˜high), and may be configured to display the user's self-understanding 2302 value at one point on the continuous spectrum graph 2301.

Referring to FIG. 5E, the service interface screen may include a region 24 that describes a rough response plan for the user by a comprehensive score range including a brain health index and a mental health index. For example, when the comprehensive score is 80 points or more, a response such as “you are in good health. maintain your current condition” is suggested, when the score is 60 to 80 points, a response such as “you need to pay attention to your health. take a short break” is suggested, and when the score is less than 60 points, a response such as “please consult a specialist” is suggested.

In addition, the service interface screen may include regions 25 and 26 representing the diagnosis results for each item of the brain health index and the mental health index. For example, the service interface screen may include graphic objects 1306, 1405, 1504, and 1605 representing the diagnosis results for brain activity, brain flexibility, brain intelligence, and brain balance, respectively, and graphic objects 1906, 2005, 2105, and 2205 representing the diagnosis results for autonomic nerve activity, autonomic nerve balance, stress state, and subjective psychological state, respectively.

Referring to FIG. 5F, the service interface screen may include regions 27 and 29 that indicate an average value of the brain health index and the mental health index based on the user's gender and age. In addition, the service interface screen may further include regions 28 and 30 that indicate where the user's brain health index and mental health index fall in a score distribution graph based on the user's age group.

In various embodiments, the processor 340 may classify the brain health index calculation results based on brain wave data for each type, and provide diagnosis results (for example, therapeutic suggestions from specialist) for each type through the service interface screen. Specifically, the processor 340 may classify the types as illustrated in [Table 1] below by matching the brain health index and physical/emotional characteristics illustrated to the user as keywords.

TABLE 1

| EEG type | Description | Therapeutic suggestions |
|---|---|---|
| Foggy | Brain wave activity is | Brain activation (reading, |
| Difficulty | significantly low, so cognitive | games, solving puzzles), |
| concentrating, | and thinking activities are hazy | cognitive training, cognitive |
| forgetfulness, | as if covered in fog, and overall | behavioral therapy, aerobic |
| helplessness, dementia | concentration is low. | exercise that makes you sweat |
| | | (more than 3 times a week, more |
| | | than 30 minutes per session) |
| Mindful | Brain wave activity is | No special suggestions |
| Meditation, | maintained high, so cognitive | |
| mindfulness, self- | and thinking activities are | |
| regulation, control, | regulated to a state where | |
| sleepiness | excitement is excluded, and a | |
| | calm state is maintained. | |
| Talented | Brain wave activity is | No special suggestions |
| Concentration, | maintained high, so not only is | |
| intelligence, smartness, | the concentration high in | |
| gifted | cognitive and thinking activities, | |
| | but clarity and sparkling | |
| | creativity stand out. | |
| Tension | Brain wave activity is | Mindfulness training, rest, |
| Burden, tension, | significantly high, so cognitive | zoning out (fishing, staring at a |
| compulsion, insomnia | and thinking activities are | fire, staring at water, looking at |
| | overloaded, and they are overly | the sea), progressive muscle |
| | aroused and sensitive to various | relaxation training, cognitive |
| | stimuli. | behavioral therapy, light |
| | | walking (more than 3 times a |
| | | week, more than 30 minutes per |
| | | session) |
| Foggy-tense | Brain wave activity is irregularly | Rest, mindfulness training, |
| Burden, tension, | repeated between high and low, | stability, light brain activation |
| difficulty concentrating, | so cognitive and thinking | (reading, games, solving |
| forgetfulness | activities frequently alternate | puzzles), cognitive training, light |
| | between hyperactivity and | walking, and running (more than |
| | hypoactivity, so concentration is | 3 times a week, more than 30 |
| | not maintained consistently. | minutes per session), behavioral |
| | | activation |
| Stable | Brain wave activity is | No special suggestions |
| Stability, balanced, | appropriate, so cognitive and | |
| healthy | thinking activities remain | |
| | normally activated, overall | |
| | concentration is maintained and | |
| | higher-order cognition is | |
| | possible. | |

In various embodiments, the processor 340 may classify the results of calculating the mental health index based on the heartbeat data for each type, and provide the diagnosis results (for example, therapeutic suggestions from specialist) for each type through the service interface screen. Specifically, the processor 340 may classify the types as illustrated in [Table 2] below by matching the mental health index and the physical/emotional characteristics illustrated to the user as keywords.

TABLE 2

| PPG type | Description | Therapeutic suggestions |
|---|---|---|
| Mind weak | The autonomic nervous system | Rest, travel, mindfulness |
| Palpitations, shortness of | is generally weak, so the ability | training, light walking, |
| breath, depression, | to cope with changes in the | behavioral activation, |
| sweating, indigestion, | external environment and stress | supportive counseling, |
| headaches | is relatively low. | aerobic exercise |
| Weak-depressive | The state is a state of lethargy in | Stability, rest, light walking, |
| Decreased energy, | which the autonomic nervous | supportive counseling, |
| lethargy | system is generally weak, | aerobic exercise, mindfulness |
| | physiological balance is broken, | training, behavioral |
| | and the ability to cope with | activation, meeting a |
| | changes in the external | psychiatrist |
| | environment and relieve stress is | |
| | reduced. | |
| Weak-anxious | The autonomic nervous system | Stability, rest, light walking, |
| Excessive anxiety, tension, | is generally weak and | progressive muscle relaxation |
| and impulsiveness | physiologically unbalanced, and | training, mindfulness |
| | is very sensitive to changes in | training, cognitive behavioral |
| | the external environment and | therapy, behavioral |
| | stress, causing excessive tension. | activation, and meeting a |
| | | psychiatrist |
| Anxious | The physiological balance of the | Stability, rest, progressive |
| Anxiety, tension | autonomic nervous system is | muscle relaxation training, |
| | disrupted, making it easily | mindfulness training, |
| | affected by the external | cognitive behavioral therapy, |
| | environment and stress, | light walking, and meeting a |
| | accompanied by acute | mental health specialist |
| | psychogenic symptoms such as | |
| | anxiety and excitement. | |
| Depression | The physiological balance of the | Stability, rest, light walking, |
| Depression, lethargy | autonomic nervous system is | aerobic exercise, mindfulness |
| | disrupted, leading to a decline in | training, cognitive behavioral |
| | overall health and bioenergy | therapy, behavioral |
| | accumulated by the external | activation, meeting a mental |
| | environment and stress, and | health specialist |
| | persistent depression. | |
| Stable | The autonomic nervous system | No special suggestions |
| Stability, balance, health | is in physiological balance, so it | |
| | responds quickly and | |
| | appropriately to the external | |
| | environment and stress, and is | |
| | stably regulated. | |

So far, the mental health information providing device 300 of the present disclosure and the user interface providing method for providing mental health information performed by the processor 340 of the mental health information providing device 300 have been roughly explained. According to the present disclosure, by numerically expressing a person's mental and mental state as a brain health index and a mental health index, it can help a medical professional's quick evaluation (diagnosis), and further, it can be utilized as a means for preventing diseases and accidents (for example, health checkup records for insurance subscription) according to the user's current state. In addition, by simplifying and expressing a professional diagnosis region as a graph, it can help the user's understanding of the current mental health state. Although the embodiments of the present disclosure have been described in more detail with reference to the attached drawings, the present disclosure is not necessarily limited to these embodiments, and can be variously modified and implemented within a scope that does not depart from the technical idea of the present disclosure. Therefore, the embodiments disclosed in the present disclosure are not intended to limit the technical idea of the present disclosure, but to explain, and the scope of the technical idea of the present disclosure is not limited by these embodiments. Therefore, it should be understood that the embodiments described above are exemplary in all respects and not restrictive. The protection scope of the present disclosure should be interpreted by the claims below, and all technical ideas within the scope equivalent thereto should be interpreted as being included in the scope of the rights of the present disclosure.

1. A method performed by a processor of a mental health information providing device for providing a user interface for providing mental health information, the method comprising:
obtaining brain wave data and heartbeat data of a user;
calculating a brain health index representing brain activity, brain flexibility, brain intelligence, and brain balance based on the brain wave data and calculating a mental health index representing autonomic nerve activity, autonomic nerve balance, and stress based on the heartbeat data; and
providing a service interface screen that graphically visualizes the calculated brain health index and mental health index.

2. The method of claim 1, wherein the service interface screen includes a region that displays an activation level of each frequency band of delta wave, theta wave, low-alpha, high-alpha, low-beta, middle-beta, high-beta, gamma calculated based on the brain wave data as a graph for the brain activity.

3. The method of claim 2, wherein the service interface screen includes a region where the brain activation level by each frequency band is displayed in different colors in the graph for the brain activity and a normal range region is displayed on a reference axis representing the brain activation level by each frequency band.

4. The method of claim 2, wherein the calculating includes further includes comparing the brain activation level of each of a plurality of frequency bands with a preset reference value, and generating a brain-related diagnosis result of the user based on a comparison result, and the interface screen includes a region representing the diagnosis result in a region adjacent to the graph for the brain activity.

5. The method of claim 1, wherein the service interface screen includes a region that represents connection strength between the brain regions calculated based on the brain wave data as a continuous spectrum graph for the brain flexibility.

6. The method of claim 5, wherein the service interface screen includes a region where the connection strength is displayed at one point in the continuous spectrum corresponding to the brain flexibility, and brain network images corresponding to reference values of the continuous spectrum are displayed.

7. The method of claim 1, wherein the service interface screen includes a region where a peak value of any one frequency band calculated based on the brain wave data is represented as a graph for the brain intelligence in a quadrant representing a frequency and an output value (μV²) of the frequency.

8. The method of claim 1, wherein the service interface screen includes a region that represents a prefrontal activation asymmetry index calculated based on the brain wave data as a semicircular scale graph for the brain balance.

9. The method of claim 8, wherein the service interface screen includes a region that is configured such that the prefrontal activation asymmetry index points to a point on a semicircular scale corresponding to left and right prefrontal activation levels and that behavioral characteristic information according to asymmetry on the semicircular scale is displayed at both ends.

10. The method of claim 1, further comprising, after the obtaining, extracting heart rate variability (HRV) data for calculating the mental health index based on the heartbeat data.

11. The method of claim 10, wherein the service interface screen includes a region that displays an activation level of each component of a very low frequency (VLF), a low frequency (LF), a high frequency (HF), and total power (TP) (including VLF, LF, and HF) calculated based on the heart rate variability data as a graph for the autonomic nerve activity.

12. The method of claim 11, wherein the service interface screen includes a region where the activation level is displayed in different colors in the graph for the autonomic nerve activity and a normal range region is displayed on a reference axis representing the activation level.

13. The method of claim 11, wherein the service interface screen includes a region where the diagnosis result related to the autonomic nerve activity is displayed in a region adjacent to the graph for the autonomic nerve activity.

14. The method of claim 10, wherein the service interface screen includes a region that displays sympathetic and parasympathetic nerve activity calculated based on the heart rate variability data as a percentage ratio in a comparison graph with respect to the autonomic nerve balance.

15. The method of claim 14, wherein the service interface screen includes a region where each percentage ratio in the comparison graph for the autonomic nerve balance is displayed as a bar scale, and abnormal phenomenon information according to imbalance of the sympathetic and parasympathetic nerve activity is displayed at both ends.

16. The method of claim 10, wherein the service interface screen includes a region that displays a stress index and stress resistance calculated based on the heart rate variability data as a continuous spectrum graph for the stress state.

17. The method of claim 16, wherein the service interface screen includes a region where the stress index and the stress resistance are displayed at one point in a continuous spectrum corresponding to the stress state.

18. The method of claim 10, further comprising:
before the calculating, obtaining psychological test result data of the user, and
the service interface screen includes a region that represents a numerical value corresponding to the test result data as a continuous spectrum graph for subjective psychological state.

19. The method of claim 18, wherein the calculating further includes generating a self-understanding value of the user based on the heart rate variability data and the psychological test result data, and the service interface screen includes a region that a continuous spectrum graph for self-understanding is placed in a region adjacent to a graph related to the mental health index, and the self-understanding value is displayed at one point in the continuous spectrum.

20. The method of claim 1, wherein the service interface screen includes a region where graphic objects representing diagnosis results of each of the brain health index representing the brain activity, brain flexibility, brain intelligence, and brain balance, and the mental health index representing the autonomic nerve activity, autonomic nerve balance, stress, and subjective psychological state are displayed.

21. A device comprising:
a communication interface;
a memory; and
a processor operably connected to the communication interface and the memory,
wherein the processor is configured to obtain brain wave data and heartbeat data of a user, calculate a brain health index representing brain activity, brain flexibility, brain intelligence, and brain balance based on the brain wave data, calculate a mental health index representing autonomic nerve activity, autonomic nerve balance, and stress based on the heartbeat data, and provide a service interface screen that graphically visualizes the calculated brain health index and mental health index.
